(19) United States
(12) Patent Application Publication
(10) Pub. No.: US 2010/0094246 A1
(43) Pub. Date: Apr. 15, 2010

(54) LONG-TERM FEED - ELDERLY

(76) Inventors: Herve Le-Henand, Saint Germain en Laye (FR); François Murbach, Divonne-les-Bains (FR); Michael Jedwab, Lausanne (CH); Claudia Roessle, Morges (CH); Luc Cynober, Paris (FR); Agathe Raynaud-Simon, Irvy/Seine (FR)

Correspondence Address: Nestle HealthCare Nutrition, 12 Vreeland Road, 2nd Floor, Box 697, Florham Park, NJ, 07932, US

(21) Appl. No.: 12/444,936

(22) PCT Filed: Oct. 17, 2007
(86) PCT No.: PCT/EP07/61098

§ 371 Date: Dec. 9, 2009

Related U.S. Application Data
(60) Provisional application No. 60/862,156, filed on Oct. 19, 2006.

(51) Int. Cl. A23L 1/302; A23L 1/30; A23L 1/29; A23L 1/305; A23L 1/308; A23L 1/304; A61M 39/00

(52) U.S. Cl. 604/500; 426/72; 426/73

(57) ABSTRACT

The present invention provides methods of providing long-term and tube-fed nutrition to patients requiring same. More specifically, the present invention provides methods and compositions for providing long-term nutrition to an elderly patient.

### BACKGROUND

The present application relates to nutrition. More specifically, the present invention relates to clinical nutrition.

Due to a variety of diseases, insults, and complications, patients may not be able to obtain the necessary nutrition by ingesting food through the mouth, e.g., eating food. Therefore, it has been known to provide clinical nutrition either enterally or parenterally. A variety of different formulations have been developed to provide such clinical nutrition.

Even with respect to typical enteral nutritional products, these products are designed for short-term use, typically 10 to 24 days. In this regard, the products usually provide the essential nutritional components to provide necessary nutrition to patients having acute pathologies during their hospital stays. Although these products are suitable for such short term use, they have not necessarily been designed for long-term feeding of patients. With advances in medicine resulting in increased life expectancy and better disease treatments, a number of individuals could benefit from products designed to provide long-term enteral nutrition.

### SUMMARY

The present invention provides methods of providing long-term tube-fed nutrition to patients requiring same. More specifically, the present invention provides methods and compositions for providing long-term tube-fed nutrition to an elderly patient.

To this end, in an embodiment, the present invention provides a method for providing tube-fed nutrition to an elderly patient comprising the steps of: administering long term, at least once a day, through a tube, a nutritional product comprising: a source of protein that comprises 14 to 20% by caloric content of the product; a source of carbohydrates that comprises 10 to 50% by caloric content of the product; a source of lipids that comprises 30 to 45% by caloric content of the product; a source of dietary fiber that provides 10 to 25 grams per liter of the product; and 8.0 to 18.0 micrograms of chromium per 100 kcal of the product.

Pursuant to the method, in an embodiment, the lipids can comprise 40 to 60% by weight mono-unsaturated fats (MUFA). In an embodiment, the product comprises not more than 1.1 grams per 100 kcal of saturated fatty acids (SFA) and the product comprises 0.4 to 2.0% by weight of the lipids as eicosapentaenoic acid (EPA). The protein source can be selected from the group consisting of casein, whey, and soy. The carbohydrates can be selected from the group consisting of fructose, sugar alcohols, starches, dextrins and maltodextrins.

Pursuant to the method, the product can comprise other components. For example, per 100 kcal the product can comprise at least: 30 mg choline; 4.0 mg taurine; and/or 3.0 mg carnitine. Additionally, the product can comprise per 100 kcal at least: 0.1 mg beta-carotene; 0.2 mg lycopene; and/or 0.05 mg lutein.

In an embodiment of the method, the energy density is 0.8 to 1.3 kcal/ml. If desired, the product comprises a prebiotic and provides both soluble and insoluble fibers. At least 25% by weight of the fiber can be soluble.

In another embodiment, the present invention provides a method for providing nutrition to an elderly patient comprising the steps of: administering long term, at least once a day, through a tube, a composition comprising: a protein source; a fat source; not more than 44% of the energy of the composition provided by a carbohydrate source; a source of dietary fiber comprising at least 25% by weight soluble fiber; at least 8.0 micrograms per 100 kcal of the product provided by chromium; vitamin D; and calcium.

In an embodiment of the method, the vitamin D comprises at least 0.5 micrograms per 100 kcal and the calcium comprises at least 35 mg per 100 kcal.

In an embodiment of the method, the protein source is selected from the group consisting of casein, whey, and soy and the carbohydrates are selected from the group consisting of fructose, sugar alcohols, starches, dextrins and maltodextrins.

Still further, pursuant to the present invention, a composition for providing long-term tube-fed nutrition is provided comprising: a source of protein that comprises 14 to 20% by caloric content of the product; a source of carbohydrates that comprises 10 to 50% by caloric content of the product; a source of lipids that comprises 30 to 45% by caloric content of the product; a source of dietary fiber that provides 10 to 25 grams per liter of the product and comprising both soluble and insoluble fibers, wherein at least 25% by weight of the fiber is soluble; 8.0 to 18.0 micrograms of chromium per 100 kcal of the product.

A number of variations and additions to the product are possible. For example, the lipids can comprise 40 to 60% by weight mono-unsaturated fats, not more than 1.1 grams per 100 kcal of saturated fatty acids, and 0.4 to 2.0% by weight of the lipids as eicosapentaenoic acid. The protein source can be selected from the group consisting of casein, whey, and soy and the carbohydrates can be selected from the group consisting of fructose, sugar alcohols, starches, dextrins and maltodextrins. In an embodiment, the product comprises per 100 kcal at least: 30 mg choline; 4.0 mg taurine; and 3.0 mg carnitine. In another embodiment, the product comprises per 100 kcal at least: 0.1 mg beta-carotene; 0.2 mg lycopene; and 0.05 mg lutein.

An advantage of the present invention is to provide improved enteral nutrition products.

Moreover, an advantage of the present invention is to provide improved methods for providing enteral nutrition.

Furthermore, an advantage of the present invention is to provide compositions for providing long-term tube-fed nutrition to elderly patients.

Additionally, an advantage of the present invention is to provide methods of providing long-term tube-fed nutrition to elderly patients.

Additional features and advantages are described herein, and will be apparent from, the following Detailed Description.

### DETAILED DESCRIPTION

The present invention relates to clinical nutrition. More specifically, the present invention relates to providing long-term tube-fed nutrition to elderly patients. As used herein, the term “long-term” means greater than one month (30 days). As used herein, the term “tube-fed” means to provide a product to a patient through a feed tube that is received within a portion of the digestive tract of a patient, for example, a nasogastric feed tube or percutaneous endoscopic gastrostomy tube. Applicants are filing herewith a patent application entitled “METHODS OF PROVIDING LONG-TERM NUTRITION” that discloses various long-term enteral nutrition formulas and business methods based thereon, the disclosure of which is hereby incorporated herein by reference.

The long-term tube-fed nutrition products are preferably designed for elderly patients. As used herein, the term “elderly patients” refers to an adult patient sixty-five years of age or older who cannot receive nutrition through a normal diet but who is normo-metabolic (i.e. not suffering from a metabolic disorder). Such a patient may previously have undergone surgery for a cancer of the head or neck leaving an incomplete digestive tract or an inability to swallow, may have received an injury to the neck leaving him or her unable to swallow or may be unable to swallow as a result of neurological damage caused by a stroke for example, or may be suffering from a disorder such as Alzheimers, Parkinsons, dysphagia, or CVA. As used herein, the term “normal diet” means to receive at least substantially all nutrition by eating, i.e., using one's mouth, without the use of any feed tube or parenteral feed.

In the composition of the present invention, an attempt has been made to optimize the glycaemic response in elderly patients fed the formulation. Repetitive episodes of hyperglycemia is a major difficulty with enteral feeding of the elderly. This can be especially acute for those elderly patients that may require long-term tube-fed nutrition for the rest of their lives. Hyperglycemia can aggravate the chronic low-level inflammation that is thought to be associated with many symptoms of aging. For example, repeated episodes of hyperglycemia may be linked with inflammation of the endothelium of the blood vessels, leading to vascular constriction. This can lead to cardiovascular disease and problems associated therewith. Similarly, there may be increased volume of urine, leading to excessive loss of minerals and consequential load on the kidneys.

Accordingly, pursuant to the present invention, in at least an embodiment, the formulation is able to maintain metabolic status in elderly long-term tube-fed nutrition patients by maintaining glycaemic homeostasis. To this end, the composition is designed to reduce insulin resistance and/or modulate glucose response. In an embodiment, the formulation is designed to provide necessary nutrition long term at 1500 ml per day.

Preferably, the composition provides a source of protein providing 14 to 20 percent of the total caloric content of the product. Any high quality protein source can be used, or a mixture thereof, providing a balanced profile of essential amino acids. Examples include casein, whey, and soy proteins. Proteins may be intact or partially hydrolyzed. Free amino acids may be added if desired. Preferably, at least 50 percent by weight of the protein is whey protein. In an embodiment, a mixture of 60 percent whey protein and 40 percent soy protein is utilized.

Preferably, the source of carbohydrates provides 10 to 50 percent of the total caloric content of the product. In a preferred embodiment, it provides less than 45 percent of the total caloric content. The carbohydrate components are selected to preferably provide a low glycaemic response. Suitable carbohydrates are fructose, sugar alcohol such as sorbitol and xylitol, starches, dextrins, maltodextrins, and mixtures thereof.

Preferably, the source of lipids provides 30 to 45 percent of the total caloric content of which 40 to 60 percent by weight are monounsaturated fatty acids (MUFA). Any suitable mixture of dietary lipids can be used such as saturated fatty acids (SFA), MUFA, polyunsaturated fatty acids (PUFA), and medium chain triglycerides (MCT). Preferably, the saturated fatty acids do not represent greater than 1.1 g/100 kcal. Preferably, from 0.4 to 2 percent by weight of the lipids are eicosapentaenoic acid.

Additional substances can be added to the product. For example, per 100 kcal choline can be present at a level of at least 30 mg, taurine present at a level of at least 5.0 mg, and carnitine at a level of at least 3.0 mg. With respect to the phytonutrients, they can be present per 100 kcal as follows: beta-carotene at least 0.1 mg, lycopene at least 0.2 mg, and lutein at least 0.05 mg. In an embodiment, the energy density of the product is between 0.8 and 1.3 kcal/ml.

Pursuant to the formula of the present invention, by providing a decreased carbohydrate intake, and the addition of fructose, fibers, and chromium, the formula helps to maintain the glycaemic homeostasis. Moreover, the formula is able to deal with the prevalence of glucose intolerance within the patient population.

Fructose is used in an embodiment of the invention. Fructose is carried to the cells without any insulin mediation. Therefore, it is excellent source of energy alternative to glucose. In addition, fructose catalyzes the hepatic use of glucose. These two mechanisms help to avoid an excessive postprandial hyperglycemia.

The use of chromium in the composition is advantageous as it activates the oxidation of glucose. It limits the intolerance to glucose and decreases the need for insulin.

The use of modified fibers regulates the glucose absorption. This limits glycemic peaks. In addition, the vitamin D intake is high in order to maintain the bone reserves.

As noted above, the present invention provides methods as well as products that are optimized and/or improved for long-term use. In an embodiment, these product are provided to the patient outside of a hospital setting. For example, the products can be provided in a nursing home, out care patient center, or even the home of the patient. Preferably, the nutrition products are housed in a plastic bag. A variety of such bags are known, for example, 500 ml, 1000 ml, and 1500 ml bags are known in the art. It should be noted, however, that any suitable container can be used to house the nutrition product. In an embodiment, the product is designed to provide necessary nutrition at 1500 ml per day, although those skilled in the art will appreciate that variations to this level are possible.

The nutrition products are specifically designed, in an embodiment, so that they can provide complete long-term nutrition and attempt to provide the same macro and micronutrients as would be ingested by a person eating a normal diet. Thus, the formulations can mimic, in an embodiment, what is referred to herein as the 5/8 a day. As used herein, the term “5/8 a day” refers to governmental guidelines to consumers to eat five to eight helpings of fruits and vegetable per day. Thus, in an embodiment, the products are designed so that, to the extent possible, they attempt to mimic a normal diet that is preferably ingested by individuals that do not require a tube-fed product by providing micronutrients and phytonutrients found in fruit and vegetables. In an embodiment, the present invention provides a method of designing long-term enteral nutrition products based on attempting to mimic the 5/8 a day. By providing such a nutrition product, the patient's antioxidant status can be maintained as well as metabolic status. A goal being to place these patients in a state comparable, to the extent possible, to that of a completely healthy individual of the same age eating a balanced diet.

Phytonutrients have been found to provide the following characteristics: antioxidant, anti-inflammatory, detoxification, cancer protective, prevention of atherosclerosis, alleviation of metabolic syndromes, and prevention of bone loss. To achieve the necessary phytonutrients, the compositions of the present invention can include one or more of carotenoids such as lycopene (tomato), B-carotene (carrot, spinach, tomato), lutein (spinach), B-cryptoxanthin, vitamins such as mixed tocopherols (oils and nuts), and vitamin C (orange); and polyphenols such as catechins (green tea).

Preferably, the products include the necessary nutritional components to provide complete nutrition to the patient on a long-term basis. In this regard, the products include, among other possible ingredients: protein, carbohydrate, fat, vitamins, and minerals. In an embodiment, the products substantially, if not completely comply with at least certain governmental requirements. As used herein, “governmental requirements” means any recommendations from any one of the following governments: U.S., typically the USRDA, German, typically the German RDA, and French, typically the French RDA. In an embodiment, the nutrition product meets or exceeds at least one of the governmental requirements.

By way of example and not limitation, examples of the present invention will now be given.

### Example No. 1

### Elderly Product

| | | Embodiment | Embodiment |
|---|---|---|---|
| | | 1500 ml | per 100 ml |
| Calories | kcal | 1500 | 100.00 |
| Proteins | g | 60 | 4.00 |
| Whey protein | g | 36 | 2.40 |
| Soya protein isolate | g | 24 | 1.60 |
| Carbohydrates | g | 176 | 11.70 |
| Fructose | g | 16 | 1.1 |
| Maltodextrins | g | 86 | 5.8 |
| Starch | g | 57 | 3.8 |
| Carbohydrates from | g | 17 | 1.1 |
| other sources | | | |
| Fiber | g | 21 | 1.4 |
| Insoluble | % | 66 | 66 |
| Soluble | % | 34 | 34 |
| Lipids | g | 67 | 4.5 |
| SFA (includes MCT) | g | 20 | 1.3 |
| MUFA | g | 35 | 2.3 |
| PUFA | g | 10 | 0.68 |
| Linoleic acid (n-6) | g | 5.7 | 0.38 |
| α linolenic acid (n-3) | g | 1.1 | 0.07 |
| Ratio ω6/ω3 | | 3.1 | 3.1 |
| EPA | g | 0.45 | 0.03 |
| DHA | g | 0.30 | 0.02 |
| MCT | g | 10 | 0.67 |
| Minerals and Trace | | | |
| Elements | | | |
| Sodium | mg | 1185 | 79 |
| Potassium | mg | 3195 | 213 |
| Calcium) | mg | 1200 | 80 |
| Phosphorus | mg | 795 | 53 |
| Magnesium | mg | 345 | 23 |
| Chloride | mg | 1650 | 110 |
| Iron | mg | 12.3 | 0.82 |
| Zinc | mg | 15 | 1.0 |
| Copper | mg | 2.7 | 0.18 |
| Fluoride | mg | 1.2 | 0.08 |
| Chromium | μg | 225 | 15 |
| Molybdenum | μg | 195 | 13 |
| Selenium | μg | 100 | 6.7 |
| Manganese | mg | 4.0 | 0.27 |
| Iodine | μg | 150 | 10 |
| Vitamins | | | |
| Vitamin A total | IU | 4200 | 280 |
| Vitamin D | μg | 14.7 | 0.98 |
| Vitamin E | IU | 67.5 | 4.5 |
| Vitamin K | μg | 210 | 14.0 |
| Vitamin C | mg | 120 | 8.0 |
| Vitamin B1 | mg | 1.5 | 0.10 |
| (Thiamin) | | | |
| Vitamin B2 | mg | 1.95 | 0.13 |
| (Riboflavin) | | | |
| Vitamin B3-PP | mg | 15 | 1.00 |
| (Niacin) | | | |
| Vitamin B5 | mg | 8.0 | 0.53 |
| (Pantothenic acid) | | | |
| Vitamin B6 | mg | 2.25 | 0.15 |
| (Pyridoxine) | | | |
| Vitamin B8 | μg | 57 | 3.80 |
| (Biotin) | | | |
| Vitamin B9 | μg | 405 | 27 |
| (Folic Acid) | | | |
| Vitamin B12 | μg | 4.8 | 0.32 |
| Other | | | |
| Choline | mg | 795 | 53 |
| Carnitine | mg | 150 | 10 |
| Taurine | mg | 81 | 5.4 |
| Lycopene (tomato) | mg | 5.9 | 0.39 |
| Beta-carotene (carrot) | mg | 3.8 | 0.25 |
| Lutein (Spinach) | mg | 1.43 | 0.095 |

### Example No. 2

### Elderly Product

| | | Embodiment | RANGE for | Embodiment |
|---|---|---|---|---|
| | | 1500 ml | 100 kcal | per 100 ml |
| Calories | kcal | 1500 | 0.8-1.3 kcal/ml | 100.00 |
| Proteins | g | 60 | 14-20% of total | 4.00 |
| | | | energy content | |
| | | | Any balanced | |
| | | | AA profile, | |
| | | | preferably >50% | |
| | | | by weight of | |
| | | | proteins as whey | |
| | | | in partially | |
| | | | hydrolysed or | |
| | | | intact form | |
| Whey protein | g | 36 | | 2.40 |
| Soya protein | g | 24 | | 1.60 |
| isolate | | | | |
| Carbohydrates | g | 176 | 10-50% of total | 11.70 |
| | | | energy content | |
| | | | Selected to give | |
| | | | a lowered | |
| | | | glycemic | |
| | | | response, e.g. | |
| | | | glucose, sugar | |
| | | | alcohols | |
| | | | (sorbitol), | |
| | | | starches | |
| | | | (dextrins, | |
| | | | maltodextrins), | |
| | | | preferably should | |
| | | | not contain | |
| | | | sucrose | |
| Fructose | g | 16 | | 1.1 |
| Maltodextrins | g | 86 | | 5.8 |
| Starch | g | 57 | | 3.8 |
| Carbohydrates | g | 17 | | 1.1 |
| from other | | | | |
| sources | | | | |
| Fiber | g | 21 | 10-25 g/litre | 1.4 |
| Insoluble Fiber | % | 66 | | 66 |
| Soluble Fiber | % | 34 | at least 25% | 34 |
| | | | soluble | |
| | | | preferably | |
| | | | 30-55% | |
| | | | soluble | |
| Lipids | g | 67 | 30-45% of total | 4.5 |
| | | | energy content | |
| SFA (includes | g | 20 | saturated fats | 1.3 |
| MCT) | | | (not incl. MCT) | |
| | | | <10% of total | |
| | | | energy content or | |
| | | | <1.11 g/100 kcal | |
| MUFA | g | 35 | 40-60% of total | 2.32 |
| | | | lipids (by wt) | |
| PUFA | g | 10 | | 0.68 |
| Linoleic acid | g | 5.7 | | 0.38 |
| (n-6) | | | | |
| α linolenic acid | g | 1.1 | | 0.07 |
| (n-3) | | | | |
| Ratio ω6/ω3 | | 3.1 | 2 to 6 | 3.1 |
| EPA | g | 0.45 | 0.4-2% of total | 0.03 |
| | | | lipids by wt | |
| | | | (optional) | |
| DHA | g | 0.30 | | 0.02 |
| MCT | g | 10 | | 0.67 |
| Minerals and | | | | |
| Trace | | | | |
| Elements | | | | |
| Sodium | mg | 1185 | | 79 |
| Potassium | mg | 3195 | | 213 |
| Calcium) | mg | 1200 | >35 | 80 |
| Phosphorus | mg | 795 | | 53 |
| Magnesium | mg | 345 | | 23 |
| Chloride | mg | 1650 | | 110 |
| Iron | mg | 12.3 | | 0.82 |
| Zinc | mg | 15 | | 1.0 |
| Copper | mg | 2.7 | | 0.18 |
| Fluoride | mg | 1.2 | | 0.08 |
| Chromium | μg | 225 | 8-18 | 15 |
| Molybdenum | μg | 195 | | 13 |
| Selenium | μg | 100.5 | | 6.7 |
| Manganese | mg | 4.0 | | 0.27 |
| Iodine | μg | 150 | | 10 |
| Vitamins | | | | |
| Vitamin A total | IU | 4200 | | 280 |
| Vitamin D | μg | 14.7 | >0.5 | 0.98 |
| Vitamin E | IU | 67.5 | | 4.50 |
| Vitamin K | μg | 210 | | 14.0 |
| Vitamin C | mg | 120 | | 8.0 |
| Vitamin B1 | mg | 1.5 | | 0.10 |
| (Thiamin) | | | | |
| Vitamin B2 | mg | 2.0 | | 0.13 |
| (Riboflavin) | | | | |
| Vitamin B3-PP | mg | 15 | | 1.00 |
| (Niacin) | | | | |
| Vitamin B5 | mg | 8.0 | | 0.53 |
| (Pantothenic | | | | |
| acid) | | | | |
| Vitamin B6 | mg | 2.3 | | 0.15 |
| (Pyridoxine) | | | | |
| Vitamin B8 | μg | 57 | | 3.80 |
| (Biotin) | | | | |
| Vitamin B9 | μg | 405 | | 27 |
| (Folic Acid) | | | | |
| Vitamin B12 | μg | 4.8 | | 0.32 |
| Other | | | | |
| Choline | mg | 795 | If present, | 53 |
| | | | >30 mg/100 kcal | |
| Carnitine | mg | 150 | If | 10 |
| | | | present>3 mg/ | |
| | | | 100 kcal | |
| Taurine | mg | 81 | If present, >4 mg/ | 5.4 |
| | | | 100 kcal | |
| Lycopene | mg | 5.9 | >0.2 mg/100 kcal | 0.39 |
| (tomato) | | | | |
| Beta-carotene | mg | 3.8 | >0.1 mg/100 kcal | 0.25 |
| (carrot) | | | | |
| Lutein | mg | 1.43 | >0.05 mg/ | 0.095 |
| (Spinach) | | | 100 kcal | |

Pursuant to an embodiment of the present invention, the formulations of either Example No. 1 or 2 can be provided to an elderly patient who cannot eat a normal diet at least once a day on a long-term basis for as long as necessary.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for providing tube-fed nutrition to an elderly patient comprising the steps of:
administering to an elderly patient at least once a day long term through a tube a nutritional product comprising:
a source of protein that comprises 14 to 20% by caloric content of the product;
a source of carbohydrates that comprises 10 to 50% by caloric content of the product;
a source of lipids that comprises 30 to 45% by caloric content of the product;
a source of dietary fiber that provides 10 to 25 grams per liter of the product; and
8.0 to 18.0 micrograms of chromium per 100 kcal of the product.

2. The method of claim 1 wherein the lipids comprise 40 to 60% by weight mono-unsaturated fats.

3. The method of claim 1 wherein at least 25% by weight of the fiber is soluble.

4. The method of claim 1 wherein the protein source is selected from the group consisting of casein, whey, and soy.

5. The method of claim 1 wherein the carbohydrates are selected from the group consisting of fructose, sugar alcohols, starches, dextrins and maltodextrins.

6. The method of claim 1 wherein the product comprises not more than 1.1 grams per 100 kcal of saturated fatty acids.

7. The method of claim 1 wherein the product comprises 0.4 to 2.0% by weight of the lipids as eicosapentaenoic acid.

8. The method of claim 1 wherein the product comprises per 100 kcal at least:
30 mg choline;
5.0 mg taurine; and
3.0 mg carnitine.

9. The method of claim 1 wherein the product comprises per 100 kcal one or more of at least:
0.1 mg beta-carotene;
0.2 mg lycopene; and
0.05 mg lutein.

10. The method of claim 1 wherein the energy density is 0.8 to 1.3 kcal/ml.

11. The method of claim 1 wherein the source of dietary fibers provides both soluble and insoluble fibers.

12. The method of claim 1 wherein the product comprises a prebiotic.

13. A method for providing nutrition to an elderly patient comprising the steps of:
administering long term, at least once a day, through a tube, a composition comprising:
a protein source;
a fat source;
not more than 44% of the energy of the composition provided by a carbohydrate source;
a source of dietary fiber comprising at least 25% by weight soluble fiber;
at least 8.0 micrograms per 100 kcal of product provided by chromium;
vitamin D; and
calcium.

14. The method of claim 13 wherein the vitamin D comprises at least 0.5 micrograms per 100 kcal.

15. The method of claim 13 wherein the calcium comprises at least 35 mg per 100 kcal.

16. The method of claim 13 wherein the protein source is selected from the group consisting of casein, whey, and soy.

17. The method of claim 13 wherein the carbohydrates are selected from the group consisting of fructose, sugar alcohols, starches, dextrins and maltodextrins.

18. The method of claim 13 wherein the product comprises not more than 1.1 grams per 100 kcal of saturated fatty acids.

19. The method of claim 13 wherein the product comprises per 100 kcal at least:
30 mg choline;
5.0 mg taurine; and
3.0 mg carnitine.

20. The method of claim 13 wherein the product comprises per 100 kcal at least:
0.1 mg beta-carotene;
0.2 mg lycopene; and
0.05 mg lutein.

21. The method of claim 13 wherein the energy density is 0.8 to 1.3 kcal/m.

22. A composition for providing long-term tube-fed nutrition comprising:
a source of protein that comprises 14 to 20% by caloric content of the product;
a source of carbohydrates that comprises 10 to 50% by caloric content of the product;
a source of lipids that comprises 30 to 45% by caloric content of the product;
a source of dietary fiber that provides 10 to 25 grams per liter of the product and comprising both soluble and insoluble fibers, wherein at least 25% by weight of the fiber is soluble;
8.0 to 18.0 micrograms of chromium per 100 kcal of the product; and
the product comprises sufficient minerals and vitamins to meet a government regulation.

23. The product of claim 22 wherein the lipids comprise 40 to 60% by weight mono-unsaturated fats.

24. The product of claim 22 wherein the protein source is selected from the group consisting of casein, whey, and soy.

25. The product of claim 22 wherein the carbohydrates are selected from the group consisting of fructose, sugar alcohols, starches, dextrins and maltodextrins.

26. The product of claim 22 comprising not more than 1.1 grams per 100 kcal of saturated fatty acids.

27. The product of claim 22 comprising 0.4 to 2.0% by weight of the lipids as eicosapentaenoic acid.

28. The product of claim 22 comprising per 100 kcal at least:
30 mg choline;
5.0 mg taurine; and
3.0 mg carnitine.

29. The product of claim 22 comprising per 100 kcal at least:
0.1 mg beta-carotene;
0.2 mg lycopene; and
0.05 mg lutein.

30. The product of claim 22 wherein the energy density is 0.8 to 1.3 kcal/ml.
